(19) United States
(12) Patent Application Publication
(10) Pub. No.: US 2003/0135872 A1
(43) Pub. Date: Jul. 17, 2003

(54) Gene targeting methods and vectors

(76) Inventors: Robert M. Burgess JR., Temecula, CA (US); Henry H. Ji, San Diego, CA (US)

Correspondence Address: GRAY CARY WARE & FRIEDENRICH LLP, 4365 EXECUTIVE DRIVE, SUITE 1100, SAN DIEGO, CA, 92121-2133, US

(21) Appl. No.: 10/309,757

(22) Filed: Dec. 3, 2002

Related U.S. Application Data
(60) Provisional application No. 60/338,768, filed on Dec. 4, 2001.

(51) Int. Cl. A01K 67/00; A01H 1/00; C12N 15/82; C12N 5/06; C12N 5/04; C12N 15/85

(52) U.S. Cl. 800/8; 800/278; 435/455; 435/468; 435/325; 435/419

(57) ABSTRACT

Procedures and vectors are provided for the specific alteration of particular genetic loci in eukaryotic cells. One procedure utilizes fluorescence-probe-in-cell (FPIC) gene targeting DNA vectors for the purpose of creating and identifying cells which have vector sequences integrated into the host cell genome via site-specific homologous recombination. The procedure also utilizes sequences encoding in vivo detectable markers for the identification of cells which have exogenous vector sequences integrated into the genome of the host cell, either via site-specific homologous recombination or nonhomologous recombination or insertion. In addition, cells modified using a FPIC vector, and organisms generated from such cells, are provided.

The present application claims the benefit priority under 35 U.S.C. §119(e) to U.S. Serial No. 60/338,768, filed Dec. 4, 2001, the entire contents of which is incorporated herein by reference.

### BACKGROUND OF THE INVENTION

1. Field of the Invention

The present invention relates generally to the manipulation of cells for the purpose of modifying genetic loci and, more specifically, to gene targeting vectors and to methods for generating genetic modifications in cells.

2. Background Information

Stable introduction of foreign genetic material into the genomes of both prokaryotic and eukaryotic organisms has been successfully accomplished in a variety of instances for various purposes such as the expression of an exogenous gene or the disruption of an endogenous locus. It is accomplished primarily through either random genomic insertion or site-specific homologous recombination. Random integration involves the insertion of a linearized DNA fragment into the genome of the host cell at locations that are, for the most part, non-site-specific. These insertions tend to exist as multimers or concatemers and most often do not result in the disruption and inactivation of a particular locus. The possibility also exists that endogenous loci can be disrupted by the random insertion event, thus often making analysis of the effect of the exogenous gene on the cell or organism derived from the transformed cell difficult. In addition, a significant range of exogenous promoter activity can be observed depending upon the region of integration.

Insertion of DNA into the host genome via site-specific homologous recombination allows for the targeting of particular regions of the host genome for single copy integration of an exogenous DNA molecule. Homologous recombination involves the exchange of significantly similar nucleotide sequences through the function of specific recombinase enzymes. Early experiments designed to manipulate cellular endogenous genomic DNA sequences with exogenous DNA in a site-specific manner focused on yeast as a model system. Recombination was demonstrated between the yeast genome and an exogenous plasmid introduced via transformation at the leu2.sup locus (Hinnen et al. (1978), Proc. Natl. Acad. Sci. U.S.A., 75, 1929). More recently the utilization of mammalian cellular homologous recombination capacities has allowed for the generation of specific mutated DNA sequences within the cellular endogenous genomic DNA. Both gain-of-function and loss-of-function alleles have been generated in stem cells and animals generated from such cells (see below). In addition, the application of positive-negative selection vectors and methods has accelerated the generation and study of cells and animals containing mutated DNA sequences (Capecchi et al. (1997) U.S. Pat. No. 5,631,153).

A number of animals have been created from embryonic stem cells having particular loci mutated through site-specific homologous recombination. These include mice which are derived from chimeras produced by injection of blastocysts with embryonic stem cells targeted through homologous recombination at particular loci. Some examples include the p53 and paraxis loci (Donehower et al. (1992), Nature, 356, 215; Burgess et al. (1996), Nature, 384, 570). Pigs have also been derived from embryonic stem cells modified by homologous recombination include pigs (Butler et al. (2002), Nature, 415, 103).

To date, primarily two types of vectors have been designed that allow for targeting of a specific region of the genome for replacement of endogenous with exogenous DNA sequences. These vectors have proven sufficient for the generation of a variety of targeted alleles in a number of different cell types. Insertion vectors contain two regions of homology flanking an internal nucleotide sequence encoding a selectable marker. The vector is linearized within one of the regions of homology. A single crossover event and homologous recombination results in a partial duplication of genomic sequences. Intrachromosomal recombination often results in exclusion of the endogenous duplicated sequences. A disadvantage to this type of targeting vector is the lack of a negative selectable marker, which would allow for significant enrichment for correctly targeted events through elimination of cells containing backbone or vector sequences. In addition, linearization within a region of homology reduces the amount of DNA sequence available for homologous recombination thus reducing the opportunity for strand exchange (Thomas et al. (1986), Cell, 44, 49). Finally, intrachromosomal recombination must occur within a defined region or regeneration of the wild-type organization of the locus can occur.

Replacement vectors contain two regions of homology usually flanking a positive selectable marker such as the gene encoding neomycin phosphotransferase. A negative selectable maker is often located external and adjacent to one of the regions of homology to provide for enrichment of corrected targeted cell in the total population through elimination of cells containing the negative selectable cassette. Introduction of a replacement vector into cells followed by simultaneous or stepwise positive and negative selection results in the isolation of cells having about an eight- to twelve-fold enriched probability of undergoing site-specific homologous recombination due to application of the negative selectable marker. In perhaps the first successful gene targeting experiments in mammalian cells, Capecchi et al. demonstrated targeting of the mouse HPRT and int-2 loci via the use of replacement vectors (U.S. Pat. No. 5,631,153). A plethora of loci have since been successfully targeted, some by insertion vectors and the majority by replacement vectors. Many of these have included a negative selectable marker positioned external to either or both of the regions of homology, which often results in an increase in the efficiency of targeted allele identification.

Yet a number of disadvantages exist with respect to the method and utility of replacement vectors and positive-negative selection. Utilization of a number of negative selectable cassettes such as HSV thymidine kinase requires the addition of an antibiotic or selective agent such as gancyclovir, which can cause undue stress to the cells and unwanted or premature differentiation. In addition, selection of cells for enrichment with a negative selectable marker takes considerable time for recovery of the cells having resistance to the drug due to absence of the selectable marker. As well, the enrichment factors typically obtained by this methodology are at most about eight- to twelve-fold. As well, the creation of positive-negative selection vectors is often strategically difficult and time-consuming. Thus, a need exists for targeting vectors that allow site specific insertion of an exogenous nucleic acid molecule into a cell genome. The present invention satisfies this need and provides additional advantages.

### SUMMARY OF THE INVENTION

Methods are provided for the modification of genomic DNA sequences through homologous recombination of vector DNA with target DNA in eukaryotic cells (FIG. 1). The methods entail first the transformation of a cell capable of undergoing homologous recombination with a vector, referred to herein as an FPIC gene targeting vector, containing sequences substantially similar to sequences present within the genome of the cell. While the vector integration into the genome of host cells can occur in an essentially random manner, with no preference for particular regions of the genome, a certain percentage of the cells also contain the gene targeting vector integrated into the genome via site-specific homologous recombination. Subsequent selection of the transformed host cells allow for the isolation and identification of cells that have successfully undergone site-specific homologous recombination. The selection is based upon the ability to detect the presence of specific transcribed nucleotide sequences which represent sequences present within the gene targeting vector.

An FPIC vector of the invention includes a first DNA sequence, which is substantially homologous to a sequence present within the host cell genome, and a third DNA sequence, which is substantially homologous to other sequences within the host cell genome downstream or upstream of the first sequence. The vector further contains between the first and third DNA sequences, a second DNA sequence, which shares little or no homology to sequences present in the host genome and confers the ability to identify cells having vector sequences integrated into said genome. The vector also contains a fourth DNA sequence, which can be positioned either 5′ or 3′ to the first or second sequences, shares little or no homology to sequences present in the host genome, and confers a separate unique means to identify cells containing the sequences integrated into a host cell genome. It is the utilization of the combination of the second and fourth DNA sequences that allows for the identification of cells that have undergone homologous recombination of the vector with endogenous sequences. Accordingly, the present invention also provides cells transformed with such vectors, particularly cells in which homologous recombination has been mediated by such a vector, and organisms generated from such transformed cells, wherein the homologous recombination confers specific genetic alterations.

In one embodiment, the invention provides a method for identifying a transformed cell which has undergone site-specific homologous recombination utilizing an FPIC gene targeting vector. Such a method can be performed, for example, by a) transforming cells with an FPIC gene targeting vector designed to undergo site-specific homologous recombination wherein the vector includes a first DNA sequence which is substantially homologous to an endogenous genomic sequence present within the host genome; a second DNA sequence which encodes a positive selection characteristic in said cells and is non-homologous to cellular endogenous genomic sequences and therefore incapable of undergoing site-specific homologous recombination; a third DNA sequence which is substantially homologous to an endogenous genomic sequence present within the host genome and is different from the first DNA sequence; and a fourth DNA sequence which represents a specific transcribed sequence in said cells, does not encode a functional protein product and is non-homologous to a cellular endogenous genomic sequence and therefore incapable of undergoing site-specific homologous recombination; wherein the vector is capable of undergoing site-specific homologous recombination in cells through strand exchange between the first DNA sequence with endogenous target sequences and the third DNA sequence with endogenous target DNA sequences; and wherein the organization of the DNA sequences in the FPIC gene targeting vector is the first DNA sequence which is substantially homologous to target DNA sequences, the second DNA second which encodes a positive selectable marker, the third DNA sequence which is substantially homologous to target DNA sequences, the fourth DNA sequence which is transcribed but does not code for a functional protein product; b) propagating cells to select for or enrich for those which have been successfully transformed with said FPIC gene targeting vector by selecting for the presence of the positive selectable marker gene product of said second DNA sequence and for the absence of the transcribed nonfunctional sequences of said fourth DNA sequence, and c) separating cells which have said second DNA sequence encoding a positive selectable marker from cells which have said fourth DNA sequence representing transcribed sequences which do not code for a functional protein. Such a method can further include a step of characterizing the genomic DNA of said cells carrying the second DNA sequence encoding a positive selectable marker but not carrying the fourth DNA sequence encoding transcribed sequences but not encoding a functional protein product for the site-specific homologous recombination events which allow for modification of the cellular target DNA.

In another embodiment, the invention provides a method for identifying a transformed cell which has undergone site-specific homologous recombination utilizing an FPIC gene targeting vector. Such a method can be performed, for example, by a) transforming cells with an FPIC gene targeting vector designed to undergo site-specific homologous recombination wherein the vector includes a first DNA sequence which is substantially homologous to an endogenous genomic sequence present within the host genome; a second DNA sequence which encodes transcribed nucleotide sequences, does not encode a functional protein product and is non-homologous to cellular endogenous genomic sequences and therefore incapable of undergoing site-specific homologous recombination; a third DNA sequence which is substantially homologous to an endogenous genomic sequence present within the host genome and is different from the first DNA sequence; and a fourth DNA sequence which encodes a negative selectable marker and is non-homologous to a cellular endogenous genomic sequence and therefore incapable of undergoing site-specific homologous recombination; wherein the vector is capable of undergoing site-specific homologous recombination in cells through strand exchange between the first DNA sequence with endogenous target sequences and the third DNA sequence with endogenous target DNA sequences; and wherein the organization of the DNA sequences in the FPIC gene targeting vector is the first DNA sequence which is substantially homologous to target DNA sequences, the second DNA second which encodes transcribed sequences but does not encode a functional protein product, the third DNA sequence which is substantially homologous to target DNA sequences, the fourth DNA sequence which is encodes a negative selectable marker; b) propagating cells to select for or enrich for those which have been successfully transformed with said FPIC gene targeting vector by selecting for the presence of the said second DNA sequence, and c) separating cells which have said second DNA sequence encoding transcribed sequences from cells which have said fourth DNA encoding a negative selectable marker. Such a method can further include a step of characterizing the genomic DNA of said cells carrying the second DNA sequence representing transcribed sequences but not encoding a functional protein product and not carrying the fourth DNA sequence for the site-specific homologous recombination events which allow for modification of the cellular target DNA.

In yet another embodiment, the invention provides a method for identifying a transformed cell which has undergone site-specific homologous recombination utilizing an FPIC gene targeting vector. Such a method can be performed, for example, by a) transforming cells with an FPIC gene targeting vector designed to undergo site-specific homologous recombination wherein the vector includes a first DNA sequence which is substantially homologous to an endogenous genomic sequence present within the host genome; a second DNA sequence which encodes transcribed sequences but does not encode a functional protein product and is non-homologous to cellular endogenous genomic sequences and therefore incapable of undergoing site-specific homologous recombination; a third DNA sequence which is substantially homologous to an endogenous genomic sequence present within the host genome and is different from the first DNA sequence; and a fourth DNA sequence which represents a specific transcribed sequence in said cells, does not encode a functional protein product and is non-homologous to a cellular endogenous genomic sequence and therefore incapable of undergoing site-specific homologous recombination; wherein the vector is capable of undergoing site-specific homologous recombination in cells through strand exchange between the first DNA sequence with endogenous target sequences and the third DNA sequence with endogenous target DNA sequences; and wherein the organization of the DNA sequences in the FPIC gene targeting vector is the first DNA sequence which is substantially homologous to target DNA sequences, the second DNA second which represents transcribed sequences but does not code for a functional protein product, the third DNA sequence which is substantially homologous to target DNA sequences, the fourth DNA sequence which is transcribed but does not code for a functional protein product; b) propagating cells to select for or enrich for those which have been successfully transformed with said FPIC gene targeting vector by selecting for the presence of the second DNA sequence representing transcribed sequences which do not code for a functional protein product and selecting for the absence of transcribed sequences which do not code for a functional protein product from said fourth DNA sequence, and c) separating cells which have said second DNA sequence representing transcribed sequences which do not code for a functional protein product from cells which have said fourth DNA sequence representing transcribed sequences which do not code for a functional protein. Such a method can further include characterizing the genomic DNA of said cells carrying the second DNA sequence representing transcribed sequences which do not code for a functional protein product but not carrying the fourth DNA sequence encoding transcribed sequences but not encoding a functional protein product for the site-specific homologous recombination events which allow for modification of the cellular target DNA.

In still another embodiment, the invention provides a method for identifying a transformed cell which has undergone site-specific homologous recombination utilizing an FPIC gene targeting vector. Such a method can be performed, for example, by a) transforming cells with an FPIC gene targeting vector designed to undergo site-specific homologous recombination wherein the vector includes a first DNA sequence which is substantially homologous to an endogenous genomic sequence present within the host genome; a second DNA sequence which encodes transcribed sequences but does not encode a functional protein product and is non-homologous to cellular endogenous genomic sequences and therefore incapable of undergoing site-specific homologous recombination; a third DNA sequence which is substantially homologous to an endogenous genomic sequence present within the host genome and is different from the first DNA sequence; and a fourth DNA sequence which encodes a positive selectable marker and is non-homologous to a cellular endogenous genomic sequence and therefore incapable of undergoing site-specific homologous recombination; wherein the vector is capable of undergoing site-specific homologous recombination in cells through strand exchange between the first DNA sequence with endogenous target sequences and the third DNA sequence with endogenous target DNA sequences; and wherein the organization of the DNA sequences in the FPIC gene targeting vector is: the first DNA sequence which is substantially homologous to target DNA sequences, the second DNA second which represents transcribed sequences but does not code for a functional protein product, the third DNA sequence which is substantially homologous to target DNA sequences, the fourth DNA sequence which codes for a positive selectable marker; b) propagating cells to select for or enrich for those which have been successfully transformed with said FPIC gene targeting vector by selecting for the presence of the second DNA sequence representing transcribed sequences which do not code for a functional protein product and selecting for the absence of the positive selectable protein product transcribed from said fourth DNA sequences, and c) separating cells which have said second DNA sequence representing transcribed sequences which do not code for a functional protein product from cells which have said fourth DNA sequence encoding a positive selectable marker. Such a method can further include characterizing the genomic DNA of said cells carrying the second DNA sequence representing transcribed sequences which do not code for a functional protein product but not carry the fourth DNA sequence encoding a positive selectable marker for the site-specific homologous recombination events which allow for modification of the cellular target DNA.

An object of the present invention is to provide site-specific homologous recombination methods for the targeting of specific regions of eukaryotic genomes for the purposes of modifying endogenous nucleotide sequences, and compositions, including cells characterized by such site-specific homologous recombination and non-human organisms containing such cells. Accordingly, it is another object of the present invention to provide methods for the selection and detection of cells which have undergone site-specific homologous recombination. It is a further object of the present invention to provide vectors for the application of the described methods. It is yet another object of the present invention to provide nucleic acid and peptide nucleic acid (PNA) molecular beacon probes for the identification of the presence or absence of specific non-homologous gene targeting sequences in cells. It is still a further object of the present invention to provide cells which have been modified by site-specific homologous recombination methods described. It is yet another object of the present invention to provide transgenic animals and plants which have been modified by the site-specific homologous recombination and detection methods described.

Accordingly, the present invention relates to an isolated FPIC gene targeting vector for site-specific homologous recombination in cells capable of undergoing homologous recombination. Such an FPIC gene target vector includes a first DNA sequence that is substantially homologous to cellular endogenous genomic DNA sequences of target cells, and is capable of undergoing homologous recombination in the target cells; a second DNA sequence that is not homologous to cellular endogenous genomic sequences, and is not capable of undergoing homologous recombination in the genome of the target cells, the second DNA sequence further including a nucleotide sequence that is capable of allowing identification of cells containing said nucleotide sequence; a third DNA sequence that is substantially homologous to cellular endogenous genomic sequences and is capable of undergoing homologous recombination in the target cells; and, optionally, a fourth DNA sequence that is not homologous to cellular endogenous genomic sequences of the target cells, and is not capable of undergoing homologous recombination in the cells, the fourth DNA sequence, when present, further including a nucleotide sequence capable of allowing identification and separation of cells containing said DNA sequences from cells not containing said nucleotide sequence. Preferably, the component DNA sequences in the FPIC gene targeting vector are linked, in a 5′ to 3′ orientation, as follows: the first DNA sequence that is substantially homologous to cellular endogenous genomic DNA sequences, the second DNA sequence, the third DNA sequence that is substantially homologous to cellular endogenous genomic DNA sequences, and the fourth DNA sequence. Such a vector is characterized in that it can undergo site-specific homologous recombination resulting in modification of cellular endogenous target genomic DNA sequences.

As such, the present invention also relates to a method for obtaining a transformed cell that has undergone site-specific homologous recombination utilizing such a FPIC gene targeting vector. Such a method can be performed, for example, by contacting cells with an FPIC gene targeting vector as disclosed herein, under conditions suitable for transformation of the cells by the vector; propagating the cells contacted with the vector under conditions selective for the presence of transcribed sequences representing said second DNA sequence and for the absence of transcribed sequences representing said fourth DNA sequence, thereby selecting or enriching for cells transformed with said FPIC gene targeting vector; and separating cells having said second DNA sequence from cells having said fourth DNA, thereby obtaining transformed cells that have undergone site-specific homologous recombination utilizing a FPIC gene targeting vector.

### BRIEF DESCRIPTION OF THE DRAWINGS

FIG. 1 is a diagrammatic flowchart illustration of the process involved in site-directed mutagenesis employing an FPIC gene targeting vector.

FIG. 2 is a diagrammatic illustration of the molecular events of FPIC gene targeting via homologous recombination.

FIG. 3 is a diagrammatic illustration of several types of FPIC gene targeting vectors.

FIG. 4 is a diagrammatic illustration of FPIC gene targeting at the ptch2 locus.

FIG. 5 is a diagrammatic illustration of FPIC gene targeting at the paraxis locus.

### DETAILED DESCRIPTION OF THE INVENTION

The present invention is based on the development of gene targeting vectors useful for introducing modifications into cellular endogenous genomic target DNA sequences via site-specific homologous recombination. Accordingly, gene targeting vectors are provided, as are methods of using such vectors to genetically modify cells. Also provided are cells that are genetically modified using a gene targeting vector of the invention, and transgenic non-human organism generated from such cells, for example, embryonic stem (ES) cells that are genetically modified in a site-specific manner in one or more cellular endogenous genomic DNA sequence using a targeting vector of the invention, and non-human organisms derived from such modified (ES) cells.

The term “cellular endogenous genomic DNA sequence” is defined herein as a nucleotide sequence that is normally present within the genome of a cell. As disclosed herein, cellular endogenous genomic DNA sequences are capable of undergoing site-specific homologous recombination with sequences of a gene targeting vector of the invention and, therefore, can be utilized as a target for modification by the disclosed FPIC gene targeting vectors. Sequences included within this definition can represent any coding or noncoding regions of specific genes present within the cellular genome. Genes encoding such protein products as structural proteins, secreted proteins, hormones, receptors, enzymes, transcription factors are included in this definition. These sequences can also represent regulatory element identity such as promoters, enhancers or repressor elements. The organization of the cellular endogenous genomic target DNA sequence is generally similar to specific sequences present within the FPIC gene targeting vector. That is, it contains sequences which are substantially homologous to sequences present within the FPIC gene targeting vector that allow for site-specific homologous recombination to occur.

The term “site-specific homologous recombination” refers to strand exchange crossover events between DNA sequences substantially similar in nucleotide composition. These crossover events can take place between sequences contained in the FPIC gene targeting vector and cellular endogenous genomic DNA sequences. In addition, it is possible that more than one site-specific homologous recombination event can occur between DNA sequences present in the FPIC gene targeting vector and cellular endogenous genomic sequences which would result in a replacement event in which DNA sequences contained within the FPIC gene targeting vector have replaced specific sequences present within the cellular endogenous genomic sequences. As well, a single site-specific homologous recombination event can occur between DNA sequences present in the FPIC gene targeting vector and cellular endogenous genomic sequences which would result in an insertion event in which the majority or the entire FPIC gene targeting vector is inserted at a specific location within the cellular endogenous genomic sequences.

The term “first DNA sequence” refers to DNA sequences present within the FPIC gene targeting vector which are substantially homologous to cellular endogenous genomic sequences. It is these sequences which are predicted to undergo site-specific homologous recombination upon their introduction into cells capable of undergoing said recombination which contain similar sequences.

The term “second DNA sequence” refers to sequences which can be detected for their presence based upon particular characteristics and which can, but need not, be expressed independently of cellular target sequences due to the presence of absence of a promoter and regulatory elements upstream of the positive selectable marker. The second DNA sequence is positioned between the first and third DNA sequences which are substantially homologous to cellular endogenous genomic DNA sequences. The second DNA sequence is nonhomologous to cellular endogenous genomic DNA sequences and therefore incapable of site-specific homologous recombination with this sequences.

The term “third DNA sequence” refers to DNA sequences present within the FPIC gene targeting vector which are substantially homologous to cellular endogenous genomic sequences yet are different but possibly adjacent or within reasonable proximity to those of the first DNA sequence. It is these sequences which are predicted to undergo site-specific homologous recombination upon their introduction into cells capable of undergoing said recombination which contain similar sequences.

The term “fourth DNA sequence” refers to specific sequences which can be detected for their presence based upon particular characteristics and which are positioned external to the first and third DNA sequences. The fourth DNA sequence contains its own promoter and regulatory elements and therefore its expression is independent of regulatory elements present within the cellular endogenous genomic target DNA sequences. The fourth DNA sequence is nonhomologous to cellular endogenous genomic DNA sequences and therefore incapable of site-specific homologous recombination with this sequences.

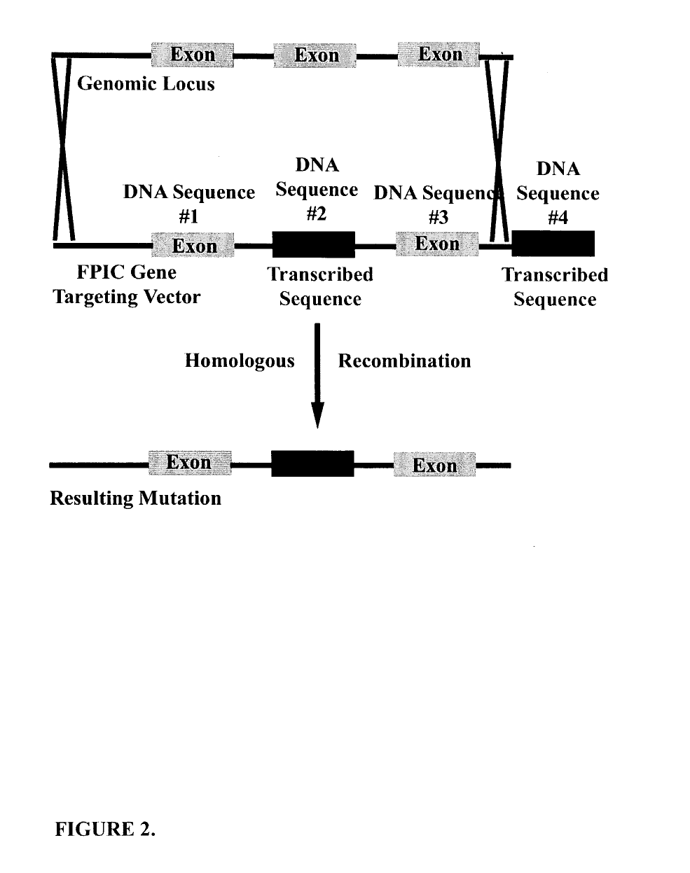

In a replacement FPIC gene targeting vector, the first, second, third and fourth DNA sequences are organized such that the second DNA sequence is positioned between the first and third DNA sequences and the fourth DNA sequence is placed either 5′ or 3′ of the first or third DNA sequences. FIG. 2 illustrates the organization of one FPIC gene targeting vector utilized for site-specific homologous recombination.

The fourth DNA sequence can be positioned either upstream or downstream of the first and third sequences contained in the FPIC gene targeting vector. Upstream generally refers to 5′ and downstream generally refers to 3′ of the first and third DNA sequences in a vector which has both the first and third DNA sequences in an orientation similar to that of cellular endogenous genomic sequences. It is to be clarified that 5′ and 3′ refer to the first and third DNA sequences respectively. This organization represents a replacement vector. It is possible that the second DNA sequence can be inverted with respect to the first and third DNA sequences and still retain expressibility and therefore allow for the ability to detect its presence. In addition, it is possible that portions of the first and third sequences in the FPIC gene targeting vector are inverted with respect to one another in comparison to similar sequences in the cellular target DNA. This type of organization represents an insertion vector. Insertion vectors generally incorporate the majority of the vector sequence into the cellular genome upon site-specific homologous recombination.

An additional selection characteristic can be represented by a fifth DNA sequence, which is placed in a position opposite that of the fourth DNA sequence. The term “opposite” refers to a position external to either the first or third DNA sequences and located on the other side of these sequences, i.e. either 5′ or 3′ in relation to the positive marker encoded by the fourth DNA sequence.

In an insertion FPIC gene targeting vector, the first, second and third sequences are organized such that the third sequence has an inverted 5′ to 3′ orientation with respect to the first sequence upon linearization of the vector. Said inverted orientation allows for the insertion of the vector at a site-specific location upon site-specific homologous recombination between the FPIC gene targeting vector and cellular endogenous genomic DNA sequences. In the majority of the cases the entire vector will be inserted and portions of the substantially homologous DNA sequences duplicated. The fourth DNA sequence encoding can, but need not, be included in the FPIC gene targeting vector.

The length of the FPIC gene targeting vector will vary depending upon the choice of positive selectable markers, the choice of nucleotide sequences which are transcribed but do not code for a functional protein product, the presence or absence of promoters capable of driving the expression of the positive selectable marker encoded by the second DNA sequence, the length of the first and third DNA sequences required for appropriate homologous recombination, the size of the base vector and the choices for selection of the plasmid vector in bacteria such as ampicillin resistance and the size of the origin of replication for the plasmid backbone. It is reasonably estimated, however, based upon the sizes of known plasmids and positive selectable markers, that the entire vector will be at least several kilobase pairs in length.

The term “molecular beacon” is defined herein as a probe for the identification of specific nucleotide sequences present within cells. The molecular beacon can be composed of nucleic acid only such as DNA or RNA, or it can be composed of a peptide nucleic acid (PNA) conjugate. Binding of the molecular beacon to specific nucleotide sequences allows for the identification of the presence of those sequences either in vitro or in vivo.

The term “functional” is defined herein with respect to positive selectable markers as conferring the ability of markers to allow for the detection and isolation of cells containing DNA encoding the positive selectable marker and to allow for the differentiation of these cells from cells which contain either no positive selectable marker or contain sequences which are transcribed but do not code for a functional protein. A number of selective agents can be utilized for the detection of positive selectable marker presence within cells. These include, but are not limited to, G418, hypoxanthine, bleomycin, hygromycin, puromycin and blasticidin, for example and are listed in Table I. In addition, positive selectable markers which do not require the addition of agents for the identification of marker presence are considered functional if they allow for the isolation of cells containing said selectable marker from cells which contain different selectable markers or no selectable marker. Some examples include, but are not limited to, the fluorescent proteins GFP, CFP, YFP, RFP, dsRED and HcRED, also listed in Table I.

TABLE I
Positive Selectable Markers Utilized in FPIC Gene targeting Vectors

| Positive Marker | Selection Agent | Method for Detection |
|---|---|---|
| NeoR | Kanamycin | Cell Survival |
| NeoR | G418 | Cell Survival |
| HygroR | Hygromycin | Cell Survival |
| hisDR | Histidinol | Cell Survival |
| GPT | Xanthine | Cell Survival |
| BleoR | Bleomycin | Cell Survival |
| HPRT | Hypoxanthine | Cell Survival |
| GFP | UV Light | Fluorescence |
| CFP | UV Light | Fluorescence |
| YFP | UV Light | Fluorescence |
| RFP | UV Light | Fluorescence |
| dsRED | UV Light | Fluorescence |
| HcRED | UV Light | Fluorescence |

The FPIC gene targeting vector includes two regions of homology, DNA sequences one and three, which are substantially homologous to regions of the host genome. Typically, the vector has lengths of homology for the first and third DNA sequences which are between about 50 base pairs and 50,000 base pairs. It also includes DNA sequences two and four, which encode a positive selectable marker, a negative selectable marker, or sequences which are transcribed that do not code for a functional protein, that allow for the identification of the presence or absence of the FPIC gene targeting vector integrant and portions thereof within the host genome. The second DNA sequence can encode for a positive selectable marker, such as, but not limited to, cyan fluorescent protein (CFP) for example, and is positioned between the two regions of homology, thus it will be included in the host genome integrant should site-specific homologous recombination occur. Ideally, the second DNA sequence will encode for a positive selectable marker if the fourth DNA sequence is transcribed but does not code for functional protein. If the second DNA sequence codes for a positive selectable marker, the fourth DNA sequence represents transcribed sequences which do not code for functional protein, and is positioned outside of the regions of homology and thus will not be incorporated into the host genome upon homologous recombination. Alternatively, if the second DNA sequence represents transcribed sequences which do not code for functional protein, the fourth DNA sequence encodes a positive selectable marker. In addition, it is possible that the second DNA sequence represents transcribed sequences that do not code for functional protein and the fourth DNA sequence represents a negative selectable marker. In addition, it is also possible that both the second and fourth DNA sequences represent transcribed sequences which do not code for functional proteins. The selection process involves sorting of cells either under a microscope or through a FACS cell sorting apparatus which will allow for the simultaneous and separate isolation of cells which contain the second DNA sequences from cells containing the fourth DNA sequences. Identification of these cells involves detection of the positive selectable marker via fluorescence or other methods and detection of the transcribed sequences which do not code for functional protein through the application of molecular beacon technologies which identify specifically transcribed sequences within cells. Cells can subsequently be propagated in tissue culture and genotyped for correct site-specific homologous recombination gene targeting events. The utilization of positive selectable markers in combination with transcribed sequences adapted to molecular beacon technology for the isolation of cells which have undergone site-specific homologous recombination allows for a substantial improvement over existing methodologies for gene targeting.

The FPIC gene targeting vectors utilized in the methods of the presently described invention are organized such that the second DNA sequence is operatively positioned between the two regions of homology and the fourth DNA sequence is operatively positioned externally or outside of the two regions of homology. It is possible that the second DNA sequence can be positioned in such a fashion as to disrupt or replace exonic or coding sequences of the endogenous region of the genome at which site-specific homologous recombination can occur thus rendering the endogenous locus inactive and thus nonfunctional.

In one aspect, the second DNA sequence can be positioned such that it replaces or inserts into regions of the genome which do not confer exonic or coding sequences such as introns, untranslated regions of exons or regulatory element regions such as promoters. In this scenario, cells can be selected that have undergone site-specific homologous recombination at the locus without inactivating that particular locus.

In another aspect, the second DNA sequence can also include regulatory elements unique to that sequence and can be positioned in such a manner that it introduces novel regulatory elements within the region of the genome selected for site-specific recombination. The invention includes the FPIC gene targeting vectors described for the purposes of performing site-specific homologous recombination and subsequent identification of cells which have undergone said recombination.

The presently described invention also includes cells which have undergone site-specific homologous recombination in accordance with the FPIC gene targeting vectors and methods for identification described herein. In addition, the presently described invention includes transgenic non-human animals which have been derived from cells which have undergone site-specific homologous recombination utilizing FPIC gene targeting vectors and methods described herein.

Also included are transgenic plants which have been derived from cells which have undergone site-specific homologous recombination utilizing FPIC gene targeting vectors and methods described herein. Plants have previously been demonstrated to undergo site-specific homologous recombination as well as gene targeting via positive-negative selection and are therefore amenable to the FPIC gene targeting vectors and methods described herein (Siebert et al. (2002), Plant Cell, 14, 1121; Hanin et al. (2001), Plant J., 28, 671; Xiaohui et al. (2001), Gene, 272, 249).

In the methods described in the present invention, the majority of the vector(s) integration into the genome of the host cell will occur in an essentially random manner, with no preference for particular regions of the genome. It is reasonably suggested, however, that a certain percentage of the first DNA vector will integrate into the genome of the host cell via site-specific homologous recombination. Subsequent selection of the cells will allow for the isolation and identification of cells which have successfully undergone site-specific homologous recombination. The selection is based upon the organization and composition of the FPIC gene targeting vector. The FPIC gene targeting vector includes two regions of homology, DNA sequences one and three, which are substantially homologous to regions of the host genome. It also includes DNA sequences two and an optional DNA sequence four, which allow for the identification of the presence or absence of the FPIC gene targeting vector integrant and portions thereof within the host genome. The second DNA sequence is positioned between the two regions of homology, thus it will be included in the host genome integrant should site-specific homologous recombination occur. The selection process involves sorting of cells either under a microscope or through a FACS cell sorting apparatus which will allow for the simultaneous and separate isolation of cells which contain the second DNA sequence from cells containing the fourth DNA sequence. Cells can subsequently be propagated in tissue culture and genotyped for correct site-specific homologous recombination gene targeting events.

The FPIC gene targeting vector method of the presently described invention is organized such that the second DNA sequence is operatively positioned between the two regions of homology. It is possible that the second DNA sequence can be positioned in such a fashion as to disrupt or replace exonic or coding sequences of the endogenous region of the genome at which site-specific homologous recombination can occur thus rendering the endogenous locus inactive and thus nonfunctional.

The second DNA sequence can be positioned such that it replaces or inserts into regions of the genome which do not confer exonic or coding sequences such as introns, untranslated regions of exons or regulatory element regions such as promoters. In this scenario, cells can be selected that have undergone site-specific homologous recombination at the locus without inactivating that particular locus.

In a third scenario, it is possible that the second DNA sequence can also include regulatory elements unique to that sequence and can be positioned in such a manner that it introduces novel regulatory elements within the region of the genome selected for site-specific recombination.

The sequence composition of the second and fourth DNA sequences are generally nonhomologous to cellular endogenous genomic DNA sequences and therefore are not capable of undergoing site-specific homologous recombination. Thus all site-specific homologous recombination is the result of the first and third DNA sequences which encode regions that are substantially homologous to cellular endogenous genomic DNA sequences and therefore capable of undergoing the strand exchange crossover process. In addition, the second and fourth DNA sequences can be positioned in an orientation-independent manner with respect to each other and with respect to the cellular endogenous genomic DNA sequences. Such a positioning for the second DNA sequence, however, requires transcription of these sequences which is independent of cellular endogenous genomic DNA regulatory elements.

A number of preferred positive selectable markers exist for use in FPIC gene targeting vectors (see Table I). These sequences allow for selection of cells carrying the positive selectable marker in order to distinguish said cells from those which do not carry the positive selectable marker. Perhaps the most widely utilized positive selectable marker utilized as the second DNA sequence encodes the neomycin phosphotransferase gene product. Other positive selectable markers appropriate for the second DNA sequence include, but are not limited to, those which code for blasticidin resistance, puromycin resistance, bleomycin resistance and hygromycin resistance. Several of these positive selectable markers can also be applied to the use of FPIC gene targeting vectors for site-specific homologous recombination in plants

The term “negative selectable marker” includes any particular gene, DNA sequence, protein, peptide or amino acid sequences which, when introduced into cells or within the proximity of cells, confers the ability to eliminate cells from a general population through the act of cell killing. The term “negative selection” refers to the act of selecting against cells through the implementation of methodologies which allow for the killing of said cells. A number of negative selectable markers can be utilized to enhance or enrich for the possibility of identifying a cell which has undergone site-specific homologous recombination. These include, but are not limited to, thymidine kinase, diphtheria toxin A chain, hprt and gpt and see Table II.

TABLE II
Negative Selectable Markers Utilized in Cotransformation Methodologies

| Negative Marker | Selection Agent | Method for Detection |
|---|---|---|
| Diphtheria Toxin | None | Cell Killing |
| Ricin Toxin | None | Cell Killing |
| HPRT | 6-Thioguanine | Cell Killing |
| HSV-Thymidine | Gancyclovir, Acyclovir, | Cell Killing |
| Kinase | FIAU | |
| GPT | 6-Thioguanine | Cell Killing |
| Cytosine Deaminase | 5-Fluoro-Cytosine | Cell Killing |

A “mutating DNA sequence” is herein referred to as any sequence which changes the nucleotide composition of cellular endogenous genomic DNA sequences. Such a change can result in an inactivation of the functional capacity of the cellular DNA sequence. Such a change can also enhance the functional capacity of the cellular DNA sequence or it can have no effect on the functional capacity of the cellular DNA sequence.

A “mutated DNA sequence” is herein referred to as any cellular endogenous genomic DNA sequence which has undergone alteration through the utilization of FPIC gene targeting vectors. It is generally anticipated that mutated DNA sequences will be generated upon site-specific homologous recombination between the FPIC gene targeting vector and cellular endogenous genomic DNA sequences. “Mutated target cells” are cells capable of undergoing site-specific homologous recombination which have a mutated DNA sequence established within the cellular genome through the application of mutating DNA sequences present in the FPIC gene targeting vectors as disclosed herein.

The term “substantially nonhomologous DNA” or “substantially not homologous DNA” refers to DNA sequences which do not contain nucleotide sequences similar enough to target DNA sequences to allow for the process of site-specific homologous recombination to occur. Dissimilar sequences of this capacity fail to undergo site-specific homologous recombination with target DNA sequences due to the mismatch of base pair composition between the two sequences.

There are a number of applicable advantages to establishing mutated DNA sequences within a cellular genome. X-linked genes, for example, can be analyzed for functional relevance in tissue culture if the particular cell type targeted by the FPIC gene targeting vector is of male origin. In addition, manipulation of embryonic stem cells via FPIC gene targeting vectors can allow for the creation of animal models for the study of human disorders. The p53 locus, for example, has been successfully inactivated via positive-negative selection technology in mouse embryonic stem cells and those cells utilized for the creation of mice deficient in the protein product encoded by this locus (Donehower et al. (1992), Nature, 356, 215). These mice are developmentally normal but susceptible to spontaneous tumors. FPIC gene targeting vectors and technology allow for the generation of similar genetic modifications in embryonic stem cells and animals created from said cells. Other uses of FPIC gene targeting vectors and technology include the generation of gain-of-function alleles, which allow for the study of a variety of cellular and physiologic phenomena. Many proto-oncogenes have been analyzed as gain-of-function alleles including c-myc, cyclin D1 and ErbB-2 (for review see Hutchinson et al. (2000), Oncogene, 19, 130). Use of the FPIC gene targeting vectors and methods described herein efficiently allow for both loss- and gain-of-function studies in embryonic stem cells as well as transgenic animals derived from these cells.

FPIC gene targeting vectors and methods are utilized for the purposes of creating and identifying cells which have undergone site-specific homologous recombination between the vector and cellular endogenous genomic target sequences. The vectors substantially enrich for the identification of cells which have undergone said process. To “substantially enrich” refers to the ability to significantly increase the likelihood of identifying cells for which site-specific homologous recombination between the vector and cell DNA sequences. The significant increase in likelihood is at least two-fold of homologous recombination events when compared to nonspecific insertion or integration events, preferably at least 10-fold, more preferably at least 100-fold and even more preferably at least 10,000-fold. Substantially enriched cell populations derived from the use of FPIC gene targeting vectors include around 1%, more preferably 10%, and even more preferably 99% of cells isolated have undergone site-specific homologous recombination between FPIC gene targeting vector sequences and cellular endogenous genomic target sequences.

It is possible that FPIC gene targeting vectors can be designed to drive the transcription of the second DNA sequences under the control of regulatory elements endogenous to the particular gene targeted by the FPIC gene targeting vector. In such an instance, the vector is constructed such that the second DNA sequences lack an upstream element sufficient to drive transcription of these sequences. Homologous recombination between the FPIC gene targeting vector and cellular endogenous genomic target sequence provides regulatory elements specific for the targeted gene which subsequently drive the transcription of the second DNA sequences. The second DNA sequences will most often not be transcribed unless site-specific homologous recombination occurs, thereby providing endogenous cellular regulatory elements sufficient to drive transcription of these sequences. An example of the organization of such a vector is to position the second DNA sequence between the first DNA sequence which is substantially homologous to cellular endogenous genomic target DNA and contains a promoter and portion of a 5′ untranslated region, and the third DNA sequence which is substantially homologous to cellular endogenous genomic target DNA and contains a portion of an intron and a downstream exon. Site-specific homologous recombination between the FPIC gene targeting vector and cellular endogenous genomic target sequences results in the positioning of the second DNA sequences under the control of endogenous regulatory elements.

A variety of scenarios are possible for the positioning of the second DNA sequence which can result in a number of phenotypes with respect to the function of the gene targeted for modification. It is possible, for example, to achieve site-specific homologous recombination between the FPIC gene targeting vector and cellular endogenous genomic target sequences without disruption of endogenous loci. This is accomplished through the positioning of the second DNA sequences within an intron or noncoding region such that introduction of said second DNA sequence into the genome of the host cell does not disrupt regulatory, exonic or coding sequences. An example of the organization of such a vector is to position the second DNA sequence between the first DNA sequence which is substantially homologous to cellular endogenous genomic target DNA and contains an exon and portion of an intron, and the third DNA sequence which is substantially homologous to cellular endogenous genomic target DNA and contains a portion of an intron and a downstream exon. Site-specific homologous recombination between the FPIC gene targeting vector and cellular endogenous genomic target sequences subsequently results in the positioning of the positive selectable marker within the intron and thus not disrupting critical exonic coding sequences. A requirement is that the second DNA sequences must be under the control of regulatory elements present within the FPIC gene targeting vector.

The introduction of a mutating DNA sequence into the genome of target cells capable of undergoing site-specific homologous recombination is not restricted to the creation of a loss-of-function or gain-of-function allele. It is possible, for example, to introduce exogenous regulatory sequences for the purposes of driving expression of particular cellular endogenous loci targeted by site-specific homologous recombination to novel tissue- and/or cell-type-specific regions within cells or transgenic animals or plants created from cells targeted by FPIC gene targeting vectors. The use of FPIC gene targeting vectors and methodologies for this purpose allows for an ability to dictate or control the spatial and temporal expression pattern of virtually any gene which is capable of undergoing site-specific homologous recombination. An example of the application of the technology described herein for this purpose would be to introduce the promoter and regulatory elements from the Pit-1 locus upstream of sequences coding for the proto-oncogene c-myc. Such a scenario would allow for ectopic expression of c-myc in somatotrope, lactotrope and thyrotrope cells of the developing and adult pituitary and provide a model for pituitary tumorigenesis (Rhodes et al. (1996), Mol. Cell Endocrinol., 124, 163; Baxter et al. (2001), 75, 9790). Table III lists a number of characterized regulatory sequences which can be utilized to drive the expression of endogenous loci through FPIC gene targeting methodologies.

TABLE III
Regulatory Element Examples.

| Regulatory Region | Expression Pattern |
|---|---|
| Pit-1 | Pituitary |
| Prolactin | Pituitary Lactotropes |
| Growth Hormone | Pituitary Somatotropes |
| Myogenin | Skeletal Muscle |
| Alpha Crystallin | Lens of the eye |
| Protamine | Testes |
| P-lim | Rathke's Pouch, motor neurons |
| GATA-3 | Liver |
| Insulin | Pancreas |
| GnRH | Hypothalamus |
| Dystrophin | Cardiac and Skeletal Muscle |

It is understood that any cell type that is capable of undergoing site-specific homologous recombination can be manipulated by FPIC gene targeting vectors and methodology for the purposes of mutating cellular endogenous genomic DNA sequences. Cells capable of undergoing site-specific homologous recombination can be derived from a variety of organisms and species including, but not limited to, human, murine, ovine, porcine, bovine, simian, canine and feline. In general, any eukaryotic cell capable of undergoing site-specific homologous recombination can be targeted successfully for the generation of a mutated DNA sequence within the cellular endogenous genomic DNA by FPIC gene targeting vectors and methodology.

When the creation of a transgenic non-human animal containing modification produced through the utilization of FPIC gene targeting vectors and methodology or cotransformation methodology is desired, the preferred cell type is embryonic stem cells. These cells are generally derived from the inner cell mass of preimplantation embryos and propagated in tissue culture for genetic manipulation. Upon mutating the cellular endogenous genomic DNA sequences through the application of FPIC gene targeting vectors and methodology, the cells are introduced into blastocysts via microinjection techniques and said blastocysts implanted into pseudopregnant female hosts (Hogan et al. (editor) (1994), Manipulating the Mouse Embryo, A laboratory manual, Cold Spring Harbor Laboratory Press, New York). Alternatively, morula aggregation methods can be implemented for the creation of embryos containing genetically modified stem cells (Kong et al. (2000), Lab Anim., 29, 25). Embryos which survive through postnatal stages often exhibit a chimeric cellular content in which a certain percentage of cells are derived from blastocyst origin and a certain percentage of cells are derived from those mutated by FPIC gene targeting vectors and technology. Chimeric animals can subsequently be bred to heterozyogosity and homozygosity for the allele mutated by FPIC gene targeting vectors and technology.

The FPIC gene targeting methodology described herein can be utilized for the purposes of correcting specific genetic defects in humans. It is possible, for example, to generate a mutated DNA sequence in human stem cells through site-specific homologous recombination between a FPIC gene targeting vector and cellular endogenous genomic DNA sequences and subsequently transplant those cells into patients for the correction of a specific genetic disorder or supplementation of a particular gene product. Another potential use for gene inactivation is disruption of proteinaceous receptors on cell surfaces. For example cell lines or organisms wherein the expression of a putative viral receptor has been disrupted using an appropriate FPIC gene targeting vector can be assayed with virus to confirm that the receptor is, in fact, involved in viral infection. Further, appropriate FPIC gene targeting vectors can be used to produce transgenic animal models for specific genetic defects. For example, many gene defects have been characterized by the failure of specific genes to express functional gene product, e.g. alpha. and .beta. thalassemia, hemophilia, Gaucher's disease and defects affecting the production of α1-antitrypsin, ADA, PNP, phenylketonurea, familial hypercholesterolemia and retinoblastemia. Transgenic animals containing disruption of one or both alleles associated with such disease states or modification to encode the specific gene defect can be used as models for therapy. For those animals which are viable at birth, experimental therapy can be applied. When, however, the gene defect affects survival, an appropriate generation (e.g. F0, F1) of transgenic animal can be used to study in vivo techniques for gene therapy.

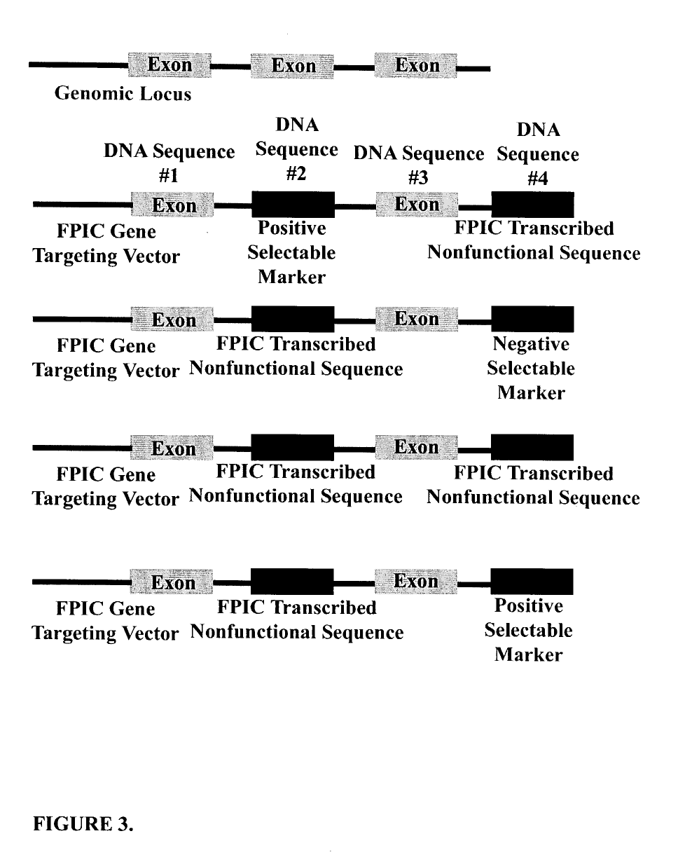

FPIC gene targeting vectors are designed for the specific purposes of mutating DNA sequences in the endogenous genomic DNA of cells capable of undergoing site-specific homologous recombination. The components of the FPIC gene targeting vector include at least one region of DNA which is substantially homologous to cellular endogenous genomic DNA sequences, one DNA sequence capable of conferring the ability to identify cells containing these sequences from cells which do not contain these sequences, at least one other DNA sequence which allows for the identification and separation of cells which contain these sequences from cells which do not contain these sequences (FIG. 3).

In addition, it is preferable that the FPIC gene targeting vector be linearized prior to its introduction into cells for the purposes of mutating cellular endogenous genomic DNA sequences as linear vectors exhibit significantly higher targeting frequencies than those circular (Thomas et al. (1986), Cell, 44, 49). It is, however, possible to successfully utilize FPIC gene targeting vectors for these purposes without linearization.

For the purposes of targeting different alleles, different regulatory elements can, but need not, be utilized for the transcription of the second and fourth DNA sequences, specifically the second DNA sequence which is positioned between the two regions of substantial homology in a replacement vector. By manipulating the levels of transcription of the second and fourth DNA sequences which are sensitive to these levels due to heterochromatic organization or adjacent regulatory elements that can affect the expression of the positive marker or can affect the process of site-specific homologous recombination can be targeted successfully. Table IV lists the more common regulatory regions that can be utilized in the presently described invention.

TABLE IV
Regulatory Regions Used to Drive Selectable Marker Expression

| Regulatory Region | Origin |
|---|---|
| Phosphoglycerate Kinase (PGK) | Mammalian |
| SV-40 (early) | Mammalian, viral |
| Cytomegalovirus (CMV) | Viral |
| Rous sarcoma virus (RSV) | Viral |
| Moloney murine leukemia virus (MMLV) | Viral |
| MC1 | Viral |

The length of the FPIC gene targeting vector required for successful site-specific homologous recombination is a critical parameter that is often dependent upon the particular gene targeted for creating a mutated DNA sequence. Vector length is dependent upon several factors. The choice of the DNA sequences representing the second and fourth DNA sequences will affect the overall vector length due to the variation of sequence composition for different transcribed or expressed DNA sequences, positive or negative selectable markers. In addition, in a replacement vector the lengths of DNA sequences one and three, the two sequences which are substantially homologous to cellular endogenous genomic target DNA sequences are crucial parameters that must be correctly addressed for successful gene targeting. In general, one region of homology can be as small as 25 bp (Ayares et al. (1986), Genetics, 83, 5199), although it is recommended that significantly larger regions of homology be utilized. Up to a certain length, an increase in the amount of homology provided in the FPIC gene targeting vector increases targeting efficiency (Zhang et al. (1994), Mol. Cell Biol., 14, 2404). In most cases the entire vector length will be a minimum of 1 kb and usually will not exceed a maximum of 500 kb, although vector length is also dependent upon the technology utilized to construct the vector. It is possible, for example, to construct a FPIC gene targeting vector with a cosmid, BAC, or YAC as the provider of the two regions of substantial homology thus generating a significantly large vector (Ananvoranich et al. (1997), BioTechniques, 23, 812; Cocchia et al., (2000), Nucleic Acids Res., 28, E81). Vector length also includes plasmid backbone sequences such as those encoding the origin of replication and bacterial drug resistance products such as ampicillin if these are not removed prior to transformation of cells with the vector.

FPIC gene targeting vector DNA sequences which are substantially homologous to cellular endogenous genomic DNA sequences and undergo site-specific homologous recombination for the purpose of creating mutated DNA sequences in cellular targets are preferred to have significantly high homology to cellular counterparts. High homology allows for efficient base pairing during the crossover and strand exchange process of site-specific homologous recombination. Any mismatch base pairing between FPIC gene targeting and cellular DNA sequences disfavors the recombination reaction. It is preferable, for example, that DNA sequences one and three in an FPIC gene targeting replacement vector are 100% homologous to cellular endogenous genomic DNA sequences, less preferable that they are 80% homologous and even less preferable that they are 50% homologous. The second and fourth DNA sequences are generally nonhomologous to cellular endogenous genomic DNA sequences and therefore do not undergo site-specific recombination with these sequences.

In certain cases it can be advantageous to remove DNA sequences which have been incorporated into the genome of cells upon site-specific homologous recombination between FPIC gene targeting vectors and cellular endogenous genomic target DNA sequences. This is due to the potential negative effects expression of these sequences can have on cellular or organismal viability and survival. Alternatively, regulatory elements introduced into the genome of the host cell can adversely affect the expression of endogenous loci juxtaposed to these elements. The removal of sequences from the host genome is possible by a number of methodologies. The Cre-Lox technology can be successfully applied for the removal of specific sequences introduced into cellular endogenous genomic DNA via FPIC gene targeting vectors and technology (for review on Cre-Lox, see Ryding et al. (2001), J. Endocrinol., 171, 1). For example, sequences encoding a positive selectable marker and corresponding regulatory elements can be flanked with Lox P recombination sites in the FPIC gene targeting vector prior to cellular transformation. After introduction of these sequences into the genome of the host cell a transient or stable expression of the Cre recombinase will allow for removal of one Lox P site and all sequences positioned between the Lox P sites. Many examples of the application of Cre-lox technology for sequence removal exist. Kaartinen et al. have demonstrated removal of a neomycin phosphotransferase cassette flanked by lox P site through the transient expression of Cre via adenoviral infection of 16-cell-stage morulae (Kaartinen et al. (2001), Genesis, 31, 126). Xu et al. successfully removed a lox P flanked neomycin phosphotransferase cassette through both a cross with mice expressing Cre under the control of the Ella promoter as well as pronuclear injection of cells containing the cassette with a Cre-expressing plasmid (Xu et al. (2001), Genesis, 30, 1). Thus, if the FPIC gene targeting vector is configured to replace or correct cellular exonic sequences that are defective, such as for human gene therapy, the transcribed sequences and corresponding regulatory elements of the second DNA sequence can be removed after completion of site-specific homologous recombination between the FPIC gene targeting vector and host DNA.

The FPIC gene targeting vectors and methodology disclosed herein can also be utilized for the purposes of mutating DNA sequences in plants. Indeed, several examples of homologous recombination in plant lineages exist (Siebert et al. (2002), Plant Cell, 14, 1121 and for review see Schaefer, (2002), Annu. Rev. Plant Physiol. Plant Mol Biol., 53, 477). In addition, said homologous recombination has been exploited utilizing positive-negative selection technology to target several plant loci including the alcohol dehydrogenase and protoporphyrinogen oxidase (PPO) loci (Xiaohui et al., (2001), Gene, 272, 249; Hanin et al., (2001), Plant J., 28, 671). It is postulated that there are a number of resistance markers which can be utilized for the purposes of implementing FPIC gene targeting methodology to generate mutated DNA sequences via site-specific homologous recombination. These include neomycin phosphotransferase as well as any herbicide or insecticide resistance loci which can allow for a positive selectable characteristic upon introduction into plant cells. Mutations in plants created utilizing FPIC gene targeting vectors and methodology can encompass loss-of-function, gain-of-function or modifications in the expression levels of endogenous loci through the introduction of exogenous regulatory elements. Loss-of-function or gain-of-function mutations can be generated through the ablation of specific endogenous DNA sequences or the alteration of sequences which can change the amino acid composition encoded by a particular plant gene. In addition, “knock-in” experiments can be performed in plants through the use of FPIC gene targeting vectors and methodology to introduce an exogenous gene or coding region into an endogenous locus.

Introduction of the FPIC gene targeting vector into plant cells can be accomplished by a variety of methods including those previously developed for the insertion of exogenous DNA into protoplasts (Hain et al. (1985), Mol. Gen. Genet., 199, 161; Negrutiu et al. (1987), Plant Mol. Biol., 8, 363; Paszkowski et al. (1984), EMBO J., 3, 2717). Microinjection can also allow for the successful introduction of the FPIC gene targeting vector into plant cells (De la Pena et al. (1987), Nature, 325, 274; Crossway et al. (1986), Mol. Gen. Genet., 202, 179). In addition, it is possible to introduce the FPIC gene targeting vector into plant cells via liposome-mediated transfection (Deshayes et al. (1985), EMBO J., 4, 2731). Upon successful introduction of the FPIC gene targeting vector into plant cells site-specific homologous recombination can allow for the mutation of cellular endogenous genomic DNA sequences according to the construction and organization of the FPIC gene targeting vector.

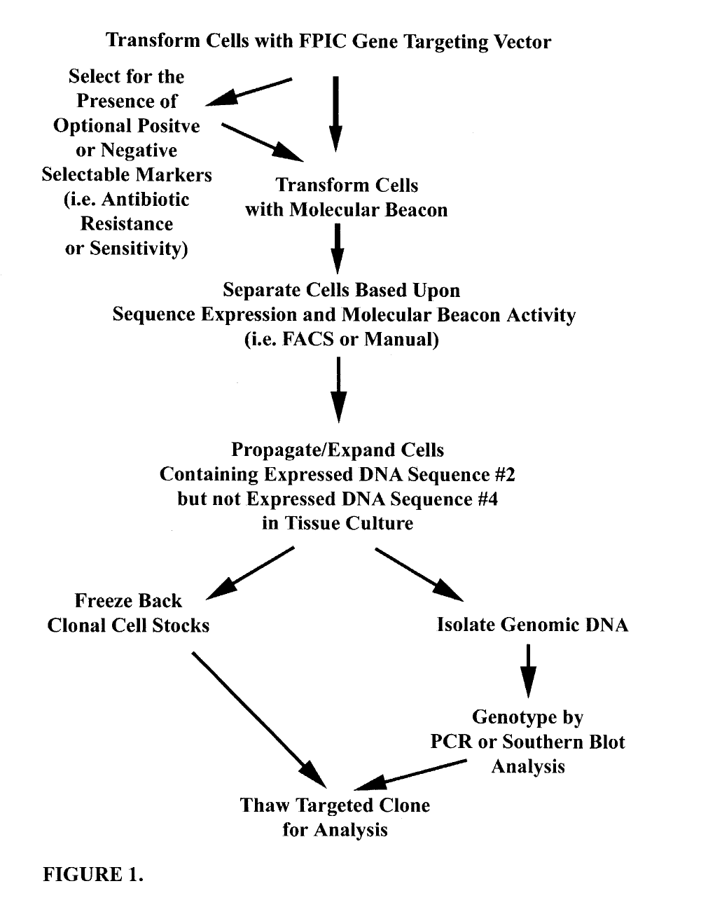

The cell separation strategies described in the present invention include cell sorting through the utilization of a FACStar Plus™ cell sorter as well as manual separation techniques, but the invention is not limited to this apparatus or to these separation techniques (FIG. 1). Other cell sorting apparatuses can also be implemented for the effective separation of cells which express specific DNA sequences verses the expression of other unique DNA sequences. These include, but are not limited to, the FACS Vantage SE I, and FACS Vantage SE II or any apparatus capable of sorting cells based upon methods described in the present invention.

The strategies for detection of the presence or absence of specific nucleotide sequences within cells in certain cases involves the use of molecular beacons. Molecular beacons are nucleic acid or peptide nucleic acid conjugates which bind to specific nucleotide sequences in vitro and/or in vivo. Often binding of molecular beacons to target nucleotide sequences results in emission of light of a specific wavelength which is dependent upon the design of the molecular beacon (Tyagi et al. (1996), Nature Biotechnol., 14, 303; Tyagi et al. (1998), Nature Biotechnol., 16, 49; Vet et al. (1999), Proc. Natl. Acad. Sci. USA, 96, 6394; Kostrikis et al. (1998), Science, 279, 1228). Molecular beacons typically consist of an oligonucleotide sequence specific for the target nucleotide sequence embedded within two complementary arm sequences. Other types of molecular beacons include those conjugated to protein or amino acid sequences often for added stability. A fluorophore such as EDANS is conjugated to the 5′ phosphate group and a quencher such as DABCYL is conjugated to the 3′ hydroxyl group. Examples of different fluorophores and their quenching efficiency by DABCYL are listed in Table V. Upon binding of the molecular beacon sequences to the target sequences the quencher and fluorophore are separated thus allowing for fluorescence of a particular wavelength. It is the fluorescence which allows for the efficient identification of the presence or absence of specific target nucleotide sequences in vitro or in vivo. Dye conjugated molecular beacon probes such as those conjugated with SybGreen™ marker can also be utilized.

TABLE V
Quenching of different fluorophores by DABCYL in molecular beacons.

| | Emission | | |
|---|---|---|---|
| | Maximum | Spectral Overlap | Quenching |
| Fluorophore | (nm) | (10⁻¹⁵ M⁻¹ cm³ | Efficiency (%) |
| Coumarin | 475 | 1.28 | 99.3 |
| EDANS | 491 | 1.12 | 99.5 |
| Fluorescein | 515 | 1.02 | 99.9 |
| Lucifer yellow | 523 | 0.87 | 99.2 |
| BODIPY | 525 | 0.85 | 95.0 |
| Eosine | 543 | 0.55 | 98.2 |
| Tetramethylrhodamine | 575 | 0.15 | 98.7 |
| Texas Red | 615 | 0.01 | 99.1 |

Molecular beacon technology is applicable to the identification of specific nucleotide sequences in living cells. Electroporation, lipofection and calcium phosphate precipitation methods have been successfully employed for the introduction of molecular beacons into living cells (Matsuo, (1998), Biochim. Biophys. Acta, 1379, 178; Sazani et al. (2001), Nucleic Acids Res., 29, 3965; Doyle et al. (2001), 40, 53; Dirks et al. (2001), Histochem. Cell Biol., 115, 3). The FPIC gene targeting strategy can employ one or all of these methods for the successful introduction of molecular beacon probes into cells for the identification of specific nucleotide sequences present within those cells.

The FPIC gene targeting vector is used in the FPIC gene targeting method to select for transformed target cells containing the second DNA sequences. Such selection procedures substantially enrich for those transformed target cells wherein homologous recombination has occurred. As used herein, “substantial enrichment” refers to at least a two-fold enrichment of transformed target cells as compared to the ratio of homologous transformants versus non-homologous transformants, preferably a 10-fold enrichment, more preferably a 1000-fold enrichment, most preferably a 10,000-fold enrichment, i.e., the ratio of transformed target cells to transformed cells. In some instances, the frequency of homologous recombination versus random integration is of the order of 1 in 1000 and in some cases as low as 1 in 10,000 transformed cells. The substantial enrichment obtained by the FPIC gene targeting vectors and methods of the invention often result in cell populations wherein about 1%, and more preferably about 20%, and most preferably about 95% of the resultant cell population contains transformed target cells wherein the FPIC gene targeting vector has been homologously integrated. Such substantially enriched transformed target cell populations can thereafter be used for subsequent genetic manipulation, for cell culture experiments or for the production of transgenic organisms such as transgenic animals or plants.

The following theoretical Examples are presented by way of example and are not to be construed as a limitation on the scope of the invention.

### EXAMPLE 1

### Inactivation Of The ptch2 Locus in ES Cells Using FPIC Gene Targeting Vectors

1. ptch2 Targeting Vector Construction

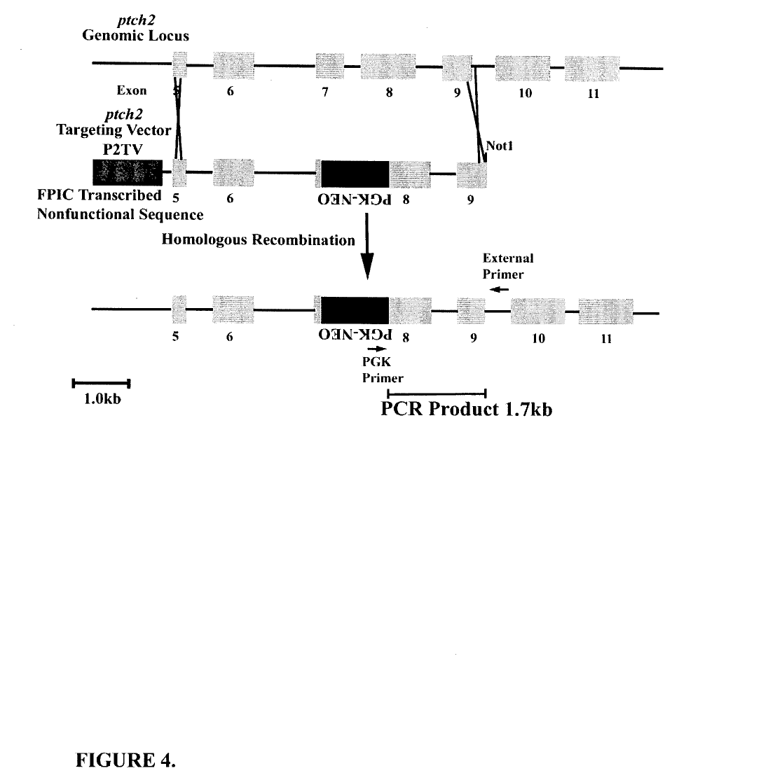

ptch2 is a transmembrane domain receptor speculated to play a role in the modulation of hedgehog signaling during embryonic development and postnatally (Motoyama et al. (1998), Nat. Genet., 18, 104; Carpenter et al., PNAS, 95, 13630, each of which is incorporated herein by reference). The ptch2 targeting vector can be constructed from a lambda phage mouse genomic DNA library utilizing a phage clone containing genomic sequences spanning exons 5 through 11, which contain transmembrane domains 2 through 8 of the ptch2 receptor (FIG. 4). Briefly, a theoretical 1.7 kb 3′ region of homology, referred to herein as the first DNA sequence, can be amplified from genomic DNA isolated from the ptch2 phage clone by PCR and flanked with Kpn1 and Not1 sites. The fragment can be subcloned into the pPOLYLINKER plasmid and the plasmid therein after referred to as pPolylinker1.7. A theoretical 5′ region of homology containing exons 5, 6 and the most 5′ region of exon 7, referred to herein as the third DNA sequence, can be removed from the genomic clone with the restriction enzymes BamH1 and Nco1, filled in with Klenow fragment DNA polymerase, and blunt subcloned into an Hpa1 site of pPolylinker1.7 to generate the plasmid therein referred to as pPolylinker1.7 upper. A DNA fragment encoding the antibiotic resistance marker neomycin phosphotransferase under the control of the phosphoglycerate kinase (PGK) promoter, referred to herein as the second DNA sequence, subsequently can be inserted between the 5′ and 3′ regions of homology to replace coding regions for transmembrane domains 2, 3 and 4, thus inactivating the receptor, and generating the plasmid designated P2. A DNA fragment encoding transcribed, but nonfunctional, sequences under the control of the cytomegalovirus (CMV) promoter, referred to herein as the fourth DNA sequence, can be subcloned upstream of the 5′ region of homology, thus generating the vector therein referred to as P2TV.

2. Transformation of ES cells with ptch2 Targeting Vector

A Not1 site present at the 3′ end of the targeting vector just downstream of the 3′ region of homology can be utilized for linearization prior to embryonic stem (ES) cell transformation. 100 μg of P2TVG vector can be linearized, phenol/chloroform extracted, ethanol precipitated and resuspended in sterile filtered water at a concentration of 1 μg/μl prior to embryonic stem cell transformation. Stem cells are propagated at 37° C., 5% CO₂ on gelatinized 10 cm plates to approximately 50% confluency in MI5 media containing 15% FCS, 0.1 mM non-essential amino acids, 1 mM sodium pyruvate, 10⁻⁴M β-mercaptoethanol, 2 mM L-glutamine, 50 μg/ml penicillin, 50 μg/ml streptomycin, 1000 U/ml LIF in Dulbecco's minimal essential medium (DMEM). Cells rinsed in serum-free DMEM can be transformed with 8 μg linearized vector per 10 cm plate of ES cells via lipofection techniques with Lipofectamine™ reagent according to the manufacturer's specifications (Invitrogen Corp.; Carlsbad Calif.). Twenty-four to 48 hours post transfection cells either can be harvested for separation in a FACStar Plus™ cell sorter or put under antibiotic selection as described below. Cell harvesting includes two rinses in sterile filtered phosphate buffered saline (PBS) followed by trypsinization in 1 ml of 0.05% trypsin/EDTA per 10 cm plate for 15 minutes. Excess trypsin is removed and cells resuspended in cell sorting buffer containing 1 mM EDTA, 25 mM HEPES, pH 7.0 and 1% dialyzed FCS in PBS at a density of 10×10⁶ cells/ml. Cells are kept on ice in 5% CO₂ prior to sorting.

3. Introduction of an Intracellular Molecular Beacon

a) ES cells transfected with P2TVG are put under selection in 200 μg/ml geneticin (G418) to select for cells that have stably incorporated the positive selectable marker neomycin phosphotransferase. Cells are selected for 12 days, and cationic lipid transfection, calcium phosphate, free uptake or electroporation procedures implemented for the introduction of molecular beacon probes, which specifically hybridize to nonfunctional sequences transcribed via control from the constitutively active promoter contained in the fourth DNA sequence. Hybridization of the molecular beacon probes to these nonfunctional transcribed sequences allows for fluorescence emission of a specific wavelength that is uniquely characteristic of the particular molecular beacon utilized for detection.

b) Alternatively, molecular beacon probes can be intracellularly introduced 48 hours post-transfection and ES cells subsequently harvested for separation via FACS cell sorting.

4. Separation of ptch2 Targeted and Nontargeted ES Cells

ES cells are harvested as described above and bulk sorted in a FACS cell sorter to separate cells that fluoresce due to specific hybridization of the fourth DNA sequence transcripts with molecular beacon probes. Sorted cell populations excluding fluorescent cells as well as unsorted cells can be replated for subsequent isolation of DNA and genotyping. Propagation of these cells can be performed under selection in 200 μg/ml geneticin (G418) to select for cells which had incorporated the positive selectable marker neomycin phosphotransferase stably.

5. Genotyping Confirmation of ptch2 Mutation by Site-Specific Homologous Recombination

Genomic DNA can be isolated from sorted ES cell populations and/or unsorted negative control cells by the following protocol. Cells are grown in 10 cm plates to approximately 80% confluence and 1 ml lysis buffer containing 100 mM sodium chloride, 50 mM Tris-HCl, pH 7.5, 10 mM EDTA and 0.5% sodium dodecyl sulfate (SDS) added directly to the plates. Cells are incubated for 15 minutes at room temperature, transferred to 1.5 ml Eppendorf tubes and incubated at 55° C. overnight with gentle shaking. Lysates are extracted two times with an equal volume of 1:1 phenol/chloroform and one time with chloroform. Genomic DNA is precipitated with an equal volume of isopropanol. After centrifugation at 15000×g genomic DNA pellets are resuspended in 300 μl sterile filtered water.

Genomic DNA from each sample can be genotyped by PCR utilizing an oligonucleotide primer specific for sequences in the PGK promoter and an oligonucleotide specific for sequences just downstream of the 3′ region of homology (FIG. 4). Twenty pmoles of each oligonucleotide are mixed with 100 ng genomic DNA in the presence of 200 μM final concentration of each dNTP, 2.5 mM MgCl₂, 1×PCR buffer and 1U Taq DNA polymerase (Invitrogen Corp.). Amplification is performed through application of the following cycling parameters: 94.0° C. for 2 minutes followed by 35 cycles of 96° C. for 30 seconds, 58° C. for 30 seconds and 72° C. for 2.5 minutes. Reactions are electrophoresed in parallel with 1 kb ladder molecular weight standards on a 0.8% agarose gel and the gel stained with ethidium bromide for UV detection of PCR products. Detection of a 1.7 kb PCR product indicates correct gene targeting through implementation of the FPIC gene targeting technology.

### EXAMPLE 2

### Inactivation Of The Paraxis Locus In ES Cells Using FPIC Gene Targeting Vectors

1. Paraxis Targeting Vector Construction

paraxis is a basic helix-loop-helix transcription factor implicated in the control of somite formation during mammalian embryogenesis (Burgess et al., (1995), 168, 296; Burgess et al., (1996), Nature, 384, 570; Barnes et al. (1997), Devel. Biol., 189, 95, each of which is incorporated herein by reference). The construction of the positive-negative selection paraxis targeting vector has been previously described (Burgess et al., supra, 1996). This vector can be modified for successful and efficient implementation of the FPIC gene targeting technology.

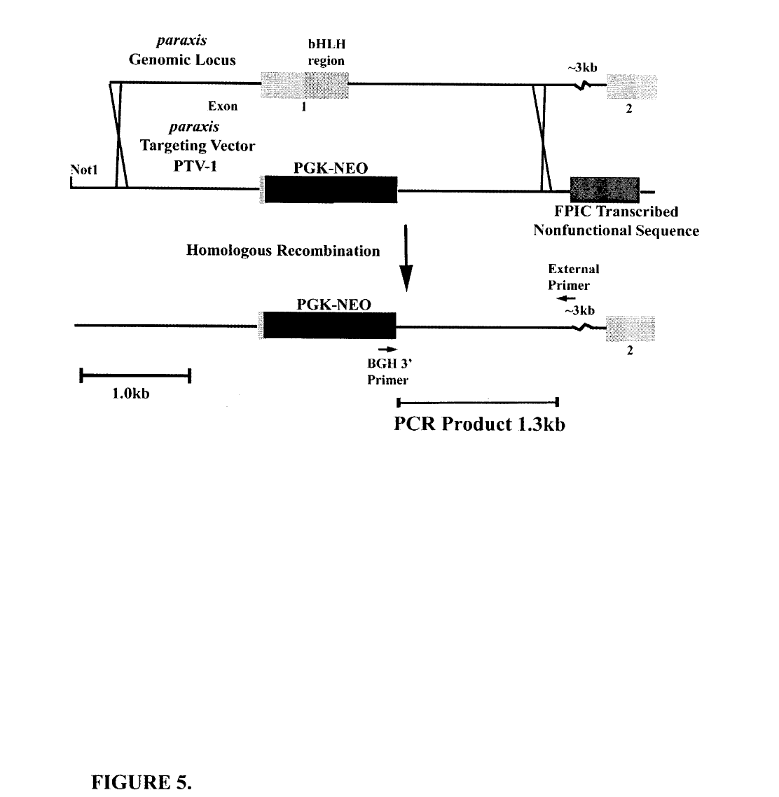

The paraxis genomic organization consists of two exons separated by a 5 kb intron (FIG. 5). The first exon contains the initiating methionine codon and the basic helix-loop-helix (bHLH) domain responsible for DNA binding and dimerization. The first exon is selected for deletion to remove sequences including the initiating methionine through the bHLH domain, thus inactivating the paraxis protein product. A 3.0 kb 5′ region of homology, referred to herein as the first DNA sequence, can be utilized for homologous recombination at the 5′ end of the paraxis locus. A 2.0 kb 3′ region of homology, referred to herein as the third DNA sequence, can be utilized for homologous recombination at the 3′ end of the paraxis locus. Neomycin phosphotransferase under the control of the PGK promoter, referred to herein as the second DNA sequence, is utilized to replace the majority of exon 1 as well as 5′ regions of intron 1 (FIG. 5). The fourth DNA sequence of the FPIC gene targeting vector is represented by the CMV promoter driving the transcription of nonfunctional sequences which specifically hybridize to molecular beacon probes. The vector containing these sequences in the described order is referred to as PTV-1.

2. Transformation of ES Cells with a Paraxis Targeting Vector

Embryonic stem cells grown are grown to approximately 50% confluency and transfected with 4 μg of linearized PTV-1. Transfections are accomplished via lipofection protocols as described above according to manufacturer's specifications (Invitrogen Corp.).

3. Introduction of an Intracellular Molecular Beacon

a) ES cells transfected with PTV-1 are put under selection in 200 μg/ml geneticin (G418) to select for cells which have incorporated the positive selectable marker neomycin phosphotransferase stably. Cells are selected for 12 days, and cationic lipid transfection, calcium phosphate precipitation, electroporation or free uptake procedures implemented for the introduction of molecular beacon probes which specifically hybridize to nonfunctional sequences transcribed via control from the constitutively active promoter contained in the fourth DNA sequence. Hybridization of the molecular beacon probes to these nonfunctional transcribed sequences allows for fluorescence emission of a specific wavelength that is uniquely characteristic of the particular molecular beacon utilized for detection.

b) Alternatively, molecular beacon probes can be intracellularly introduced 48 hours post-transfection and ES cells subsequently harvested for separation via FACS cell sorting.

4. Separation of Paraxis Targeted from Nontargeted ES Cells

Cells are harvested either with or without the implementation of antibiotic selection as described above and bulk sorted in a FACS cell sorter to separate fluorescing from nonfluorescing cells. Sorted nonfluorescing cell populations can be replated at a density of 10×10⁶ cells/10 cm plate and propagated to 80% confluency for isolation and DNA and genotyping. Such propagation can be implemented with cells under selection in 200 μg/ml geneticin (G418) to select for cells which had incorporated the positive selectable marker neomycin phosphotransferase stably.

5. Genotyping Confirmation of Paraxis Mutation by Site-Specific Homologous Recombination

Genomic DNA is isolated from either sorted ES cell populations or unsorted negative control cells as described above. Genomic DNA from each sample is subsequently genotyped by PCR utilizing an oligonucleotide primer specific for sequences in the bovine growth hormone polyadenylation signal 3′ of neo coding sequences and an oligonucleotide specific for sequences just downstream of the 3′ region of homology (FIG. 5). Reaction volumes and conditions are as described above with the exception of the primer annealing temperature which was 55° C. Reactions are electrophoresed in parallel with 1 kb ladder molecular weight standards on a 0.8% agarose gel and the gel stained with ethidium bromide for UV detection of PCR products. A 1.5 kb PCR product detected utilizing DNA from sample populations sorted to fluorescing cells indicates site-specific homologous recombination and successful gene targeting.

Although the invention has been described with reference to the above examples, it will be understood that modifications and variations are encompassed within the spirit and scope of the invention. Accordingly, the invention is limited only by the following claims.

What is claimed is:

1. A method for obtaining a transformed cell that has undergone site-specific homologous recombination utilizing a FPIC gene targeting vector, comprising:
a) contacting cells with an FPIC gene targeting vector designed to undergo site-specific homologous recombination, under conditions suitable for transformation of the cells by the vector, wherein the vector comprises:
a first DNA sequence that is substantially homologous to cellular endogenous genomic sequences and is capable of undergoing homologous recombination in said cells,
a second DNA sequence that is not homologous to cellular endogenous genomic sequences, and is not capable of undergoing homologous recombination in said cells, said second DNA sequence further comprising a nucleotide sequence that allows identification of cells containing said nucleotide sequence,
a third DNA sequence that is substantially homologous to cellular endogenous genomic sequences and is capable of undergoing homologous recombination in said cells,
a fourth DNA sequence that is not homologous to cellular endogenous genomic sequences, and is not capable of undergoing homologous recombination in said cells, said fourth DNA sequence further comprising a nucleotide sequence capable that allow identification and isolation of cells containing said nucleotide sequences from cells not containing said nucleotide sequence;
b) propagating the cells under conditions selective for the presence of transcribed sequences representing said second DNA sequence and for the absence of transcribed sequences representing said fourth DNA sequence, thereby selecting or enriching for cells transformed with said FPIC gene targeting vector; and
c) separating cells having said second DNA sequence from cells having said fourth DNA, thereby obtaining transformed cells that have undergone site-specific homologous recombination utilizing a FPIC gene targeting vector.

2. The method of claim 1, further comprising characterizing the genomic DNA of said transformed cells carrying the second DNA sequence encoding a positive selectable marker but not carrying the fourth DNA sequence encoding a transcribed selection characteristic for the site-specific homologous recombination events which allow for modification of the cellular target DNA

3. The method of claim 1 wherein said FPIC gene targeting vector comprises a positive selectable marker, which is detectable by incubating said cells in the presence of an antibiotic, or by fluorescence light emission.

4. The method of claim 1 wherein said FPIC gene targeting vector comprises a negative selectable marker, which is detectable by incubating said cells in the presence of an antibiotic.

5. The method of claim 1 wherein said FPIC gene targeting vector comprises a negative selectable marker, which is detectable without using an antibiotic or drug.

6. The method of claim 1, wherein said cells are capable of homologous recombination.

7. The method of claim 1, wherein said cells are cells of a multicellular organism.

8. The method of claim 1, wherein said cells are plant cells.

9. The method of claim 1, wherein said cells have undergone multiple rounds of site-specific homologous recombination for the purposes of multiple modifications of the endogenous cellular genome.

10. The method of claim 1, wherein said transformed cells are utilized to generate a multicellular organism.

11. The method of claim 1, wherein said cells are embryonic stem cells.

12. An isolated fluorescence-probe-in-cell (FPIC) gene targeting vector for site-specific homologous recombination in cells capable of undergoing homologous recombination, the vector comprising:
a first DNA sequence that is substantially homologous to cellular endogenous genomic sequences and is capable of undergoing homologous recombination in said cells,
a second DNA sequence that is not homologous to cellular endogenous genomic sequences, and is not capable of undergoing homologous recombination in said cells, said second DNA sequence further comprising a nucleotide sequence capable of allowing identification of cells containing said nucleotide sequence,
a third DNA sequence that is substantially homologous to cellular endogenous genomic sequences and is capable of undergoing homologous recombination in said cells,
a fourth DNA sequence that is not homologous to cellular endogenous genomic sequences, and is not capable of undergoing homologous recombination in said cells, said fourth DNA sequence further comprising a nucleotide sequence capable of allowing identification and separation of cells containing said DNA
sequences from cells not containing said nucleotide sequence.
said FPIC gene targeting vector comprising, in 5′ to 3′ orientation, the first DNA sequence that is substantially homologous to cellular endogenous genomic DNA sequences, the second DNA sequence, the third DNA sequence that is substantially homologous to cellular endogenous genomic DNA sequences, and the fourth DNA sequence; and
wherein said vector is capable of undergoing site-specific homologous recombination resulting in modification of cellular endogenous target genomic DNA sequences.

13. The FPIC gene targeting vector of claim 12, wherein said cellular endogenous genomic target DNA comprises exons and introns.

14. The FPIC gene targeting vector of claim 13, wherein said vector contains all or portions of said exons and introns, which are substantially homologous to cellular target genomic DNA sequences.

15. The FPIC gene targeting vector of claim 12, wherein said vector contains all or portions of regulatory elements, which are substantially homologous to cellular target genomic DNA sequences.

16. The FPIC gene targeting vector of claim 12, wherein said vector contains alterations in the sequences that are substantially homologous to cellular target genomic DNA sequences, said alterations comprising deletions, substitutions, additions, point mutations, or a combination thereof.

17. The FPIC gene targeting vector of claim 12, wherein said second DNA sequence encodes a transcribed nonfunctional selection characteristics in said cells and is non-homologous to a cellular endogenous genomic sequence and therefore incapable of undergoing site-specific homologous recombination.

18. The FPIC gene targeting vector of claim 12, wherein said fourth DNA sequence encodes a transcribed nonfunctional selection characteristics in said cells and is non-homologous to a cellular endogenous genomic sequence and therefore incapable of undergoing site-specific homologous recombination.

19. The FPIC gene targeting vector of claim 12, wherein said fourth DNA sequence comprises a nucleotide sequence encoding a fluorescent protein.

20. The FPIC gene targeting vector of claim 19, wherein the fluorescent protein is GFP, CFP, YFP, RFP, dsRED, or HcRED.

21. The FPIC gene targeting vector of claim 12, wherein said second DNA sequence comprises a nucleotide sequence encoding an antibiotic resistance marker.

22. The FPIC gene targeting vector of claim 21, wherein said antibiotic resistance marker is neomycin, puromycin, blasticidin, bleomycin, zeocin, or hygromycin.

23. The FPIC gene targeting vector of claim 12, wherein said second DNA sequence comprises a nucleotide sequence encoding a fluorescent protein.

24. The FPIC gene targeting vector of claim 23, wherein the fluorescent protein is GFP, CFP, YFP, RFP, dsRED, or HcRED.

25. The FPIC gene targeting vector of claim 12, wherein said fourth DNA sequence comprises a nucleotide sequence encoding a negative selection agent.

26. The FPIC gene targeting vector of claim 25, wherein the negative selection agent is thymidine kinase or diphtheria toxin A chain.

27. The FPIC gene targeting vector of claim 12, wherein said vector further comprises a fifth DNA sequence that is not homologous to cellular endogenous genomic DNA sequences, and is positioned external and 5′ to said first and third DNA sequences.

28. The FPIC gene targeting vector of claim 12, wherein each of said fourth and fifth DNA sequences encode a selectable transcribed sequence that allows for separation of cells containing DNA encoding said selectable transcribed sequences from cells that do not contain DNA encoding said selectable transcribed sequences.

29. The FPIC gene targeting vector of claim 12, wherein the substantially homologous sequence of said first DNA sequence and said third DNA sequence each is about 50 base pairs to 50,000 base pairs.

30. A cell transformed by the FPIC gene targeting vector of claim 12.

31. The cell of claim 30, wherein said vector results in the modification of at least one cellular endogenous genomic target DNA sequence.

32. The cell of claim 31, wherein said vector introduces at least one exogenous regulatory element into the cellular endogenous genomic target DNA sequence.

33. The cell of claim 30, which is an embryonic stem cell.

34. A transgenic non-human animal generated from the cell of claim 31.

35. An enriched population of cells generated by the method of claim 1, wherein said cells have undergone site-specific homologous recombination.

35. A non-human transgenic animal generated from a cell of claim 35.

36. A transgenic plant generated from a cell of claim 35.
